(19) United States
(12) Patent Application Publication
(10) Pub. No.: US 2006/0052459 A1
(43) Pub. Date: Mar. 9, 2006

(54) Antifungal medicaments comprising arylamidine derivatives

(76) Inventors: Jean-Pierre Vors, Lyon (FR); Elizabeth O'Neill, Caluire (FR); Gilbert Labourdette, Paray Le Monial (FR); Gillian Mansfield, Lyon (FR); John Pillmoor, Yorkshire (GB); Thierry Barchietto, Puiseux En (FR)

Correspondence Address: OSTROLENK FABER GERB & SOFFEN, 1180 AVENUE OF THE AMERICAS, NEW YORK, NY, 100368403, US

(21) Appl. No.: 10/532,033

(22) PCT Filed: Oct. 24, 2003
(86) PCT No.: PCT/EP03/13335

§ 371 Date: Aug. 9, 2005

(30) Foreign Application Priority Data
Oct. 24, 2002 (EP) 02356210.1

(51) Int. Cl. A61K 31/155; A61K 31/7072; A61K 31/7048; A61K 31/496; A61K 38/16

(52) U.S. Cl. 514/637; 514/28; 514/49; 514/254.07; 514/2

(57) ABSTRACT

The subject of the present invention is novel antifungal medicaments based on N2-phenylamidine derivatives and optionally at least one antifungal synergistic agent.

The subject of the present invention relates to novel antifungal medicaments.

More precisely, the subject of the present invention concerns novel antifungal medicaments based on N₂-phenylamidine derivatives and optionally at least one other synergistic antifungal agent.

The expression antifungal medicament is understood to mean a pharmaceutical composition intended to be administered to a human being or an animal.

International application WO-00/46184 describes one or more N₂-phenylamidine derivatives. Such compounds are used in the agricultural field as antifungal agents.

The applicant has demonstrated quite unexpectedly that N₂-phenylamidine derivatives also constituted antifungal compounds of choice, both in human being and in animal.

Accordingly, one of the main objectives of the present invention is to provide a novel antifungal medicament based on N₂-phenylamidine derivatives.

Another main objective of the invention is to provide a completely effective novel antifungal medicament, especially as regards its efficacy against fungi.

Another main objective of the invention is to provide a novel fungicidal medicament synergistically combining at least one N₂-phenylamidine derivative and at least one other compound known as having an antifungal activity in human being or in animal.

Another main objective of the invention is to provide a novel broad-spectrum antifungal medicament.

Another main objective of the invention is to provide a novel antifungal medicament as defined in the above objectives and which is useful in the preventive and curative treatment of fungal diseases, in particular Candida albicans and Aspergillus fumigatus infections.

All these objectives, among others, have been achieved by the inventors who have had the merit of finding that N₂-phenylamidine derivatives surprisingly and unexpectedly exhibited a very high and perennial antifungal efficacy against a broad spectrum of agents which are infectious to human being or to animal.

The present invention, which totally or partially satisfies the above-mentioned objectives, therefore relates firstly to an antifungal medicament, characterized in that it comprises at least one compound of formula (I): 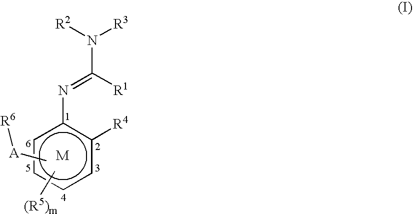
in which:
R¹ is an alkyl, an alkenyl, an alkynyl, a carbocyclic or heterocydic monovalent group, it being possible for each of these groups to be substituted, or hydrogen;
R² and R³, which may be identical or different, are any one of the groups defined for R¹; a cyano; an acyl; —OR^{a} or —SR^{a}, with R^{a} corresponding to an alkyl, an alkenyl, an alkynyl, a carbocyclic or heterocyclic monovalent group, it being possible for each of these groups to be substituted, or R² and R³, or R² and R¹ may form together and with the atoms linking them, a ring which may be substituted;
R⁴ is an alkyl, an alkenyl, an alkynyl, a carbocyclic or heterocyclic monovalent group, it being possible for each of these groups to be substituted, a hydroxyl group; mercapto; azido; nitro; halo; cyano; unsubstituted or substituted acyl, amino; cyanato; thiocyanato; —SF₅; —OR^{a}; —SR^{a} or —Si(R^{a})₃;
m=0, 1, 2 or 3;
the optional R⁵ group or the optional R⁵ groups, which may be mutually identical or different, have the same definition as that given above for R⁴;
R⁶ is an unsubstituted or substituted carbocyclic or heterocyclic group; and
A is a direct bond, —O—, —S(O)ₙ—, —NR⁹—, —CR⁷═CR⁷—, —C≡C—, -A¹-, -A¹-A¹, —O-(A¹)ₖ—O—, —O—(A¹)ₖ—, -A³-, -A⁴-, -A¹O—, -A¹S(O)ₙ—, -A²-, OA²-, —NR⁹A²-, —OA²-A¹-, —OA²-C(R⁷)═C(R⁸)—, —S(O)ₙA¹-, -A¹-A⁴-, -A¹-A⁴-C(R⁸)═N—N═CR⁸—, -A¹-A⁴-C(R⁸)═N—X²—X³—, -A¹-A⁴-A³-, -A¹-A⁴-N(R⁹)—, -A¹-A⁴-X—CH₂—, -A¹-A⁴-A¹-, -A¹-A⁴-CH₂X—, -A¹-A⁴-C(R⁸)═N—X²—X³—X¹—, -A¹-X—C(R⁸)═N—, -A¹-X—C(R⁸)═N—N═CR⁸—, -A¹-X—C(R⁸)═N—N(R⁹), -A¹-X-A—X¹—, -A¹-O-A³-, -A¹—C(R⁷)═C(R⁸)—, -A¹¹—N(R⁹)-A²-N(R⁹)—, -A¹-O—N(R⁹)-A²-, -A¹-N(R⁹)-A²-N(R⁹—, -A¹-N(R⁹)-A²-, -A¹-N(R⁹)N═C(R⁸)—, -A³-A¹-, -A⁴-A³-, -A²-NR⁹—, -A¹-A²-X¹—, -A¹-A¹-A²-X¹—, —O-A²-N(R⁹)-A²-, —CR⁷═CR⁷-A²-X¹—, —C≡C-A²-X¹—, —N═C(R⁸)-A²-X¹—, —C(R⁸)═N—N═C(R⁸)—, —C(R⁸)═N—N(R⁹), —CH₂)₂O—N═C(R⁸)— or —X-A²-N(R⁹)—
with
n=0, 1 or 2,
k=1 to 9,
A¹=—CHR⁷—,
A²=—C(═X)—,
A³=—C(R⁸)═N—O—,
A⁴=—O—N═C(R⁸)—,
X=O or S,
X¹=O, S, NR⁹ or a direct bond,
X²=O, NR⁹ or a direct bond,
X³=hydrogen, —C(═O)—, —SO₂— or a direct bond,
R⁷, which are mutually identical or different, each correspond to an unsubstituted or substituted alkyl, to a cycloalkyl or a phenyl, it being possible for each of these groups to be substituted, hydrogen, a halogen, a cyano, or an acyl;
R⁸, which are mutually identical or different, each correspond to an alkyl, an alkenyl, an alkynyl, an alkoxy, an alkylthio, it being possible for each of these groups to be substituted, a carbocyclic or heterocyclic monovalent group which may be unsubstituted or substituted, or hydrogen;
R⁹, which are mutually identical or different, each correspond to an unsubstituted or substituted alkyl, to a monovalent carbocyclic or heterocyclic group which may be unsubstituted or substituted, or to an acyl; or two R⁹ groups may form together, and with the atoms linking them, a 5-7-membered ring;
the group represented on the right side of the bond A is linked to R⁶;
or -A-R⁶ and R⁵ form together with the benzene ring M, a system of unsubstituted or substituted condensed rings;
and the possible optic and/or geometric isomers, tautomers and salts, in particular addition salts with an acid or a base, which are pharmaceutically acceptable, of the derivatives of formula (I);
and mixtures thereof.

In the definitions of the compounds of formula (I) set out above, the various radicals and chemical terms used have, unless otherwise stated, the following meanings:
“alkyl or alkyl-” means a linear or branched, saturated hydrocarbon radical containing from 1 to 8 carbon atoms;
“alkenyl” means a linear or branched hydrocarbon radical containing from 1 to 8 carbon atoms and at least one unsaturation in the form of a double bond;
“alkynyl” means a linear or branched hydrocarbon radical containing from 1 to 8 carbon atoms and at least one unsaturation in the form of a triple bond;
“alkoxy” means an alkyloxy radical;
“acyl” means the formyl radical or an alkoxycarbonyl radical;
“cydoalkyl” means a saturated cyclic hydrocarbon radical containing from 3 to 8 carbon atoms;
“haloalkyl” or “haloalkyl-” means a linear or branched, saturated hydrocarbon radical containing from 1 to 8 carbon atoms and substituted with one or more halogen atoms, in particular fluorine, chlorine and bromine;
“aryl” means one or more aromatic radicals, preferably a phenyl or a naphthyl;
“heterocycle” means an unsaturated or a completely or partially saturated cyclic radical containing from 3 to 8 atoms, chosen from carbon, nitrogen, sulphur and oxygen, for example, and without limitation, pyridyl, pyridinyl, quinolyl, furyl, thienyl, pyrrolyl, oxazolinyl;
the expression ‘unsubstituted or substituted’ means that the radicals thus termed may be substituted with one or more radicals chosen from chlorine, bromine, fluorine, iodine, alkyl, alkoxy, hydroxyl, nitro, amino; cyano and acyl.

According to a preferred embodiment of the invention, the products (I) correspond to formula (I) in which:
R¹ is an alkyl, an alkenyl or an alkynyl, it being possible for each of these groups to be substituted with an alkoxy, a haloalkoxy, an alkylthiol, a halogen or a phenyl unsubstituted or substituted with an alkyl, with a haloalkyl, with an alkoxy, with a haloalkoxy, with an alkylthiol or with a halogen, or hydrogen;
R² and R³ which may be identical or different and which have the same definition as that given above for R¹ or which correspond to an alkoxy, an alkoxyalkyl, a benzyloxy, a cyano or an alkylcarbonyl;
R⁴ is an alkyl, an alkenyl or an alkynyl, it being possible for each of these groups to be substituted with an alkoxy, a haloalkoxy, an alkylthiol, a halogen or a phenyl unsubstituted or substituted with an alkyl, with a haloalkyl, with an alkoxy, with a haloalkoxy, with an alkylthiol or with a halogen; a hydroxyl; a halogen; a cyano; an acyl (preferably: —C(═O)R^{c}, —C(═S)R^{c} or —S(O)ₚR^{c}, with R^{c} corresponding to an alkyl, a haloalkyl, alkoxy, haloalkoxy, alkylthiol, an amine, a monoalkylamine, a dialkylamine or a phenyl unsubstituted or substituted with an alkyl, with a haloalkyl, with an alkoxy, with a haloalkoxy, or with an alkylthiol;
m=0 or 1;
when it is present, R⁵ is a group having the same definition as that given above for R^{4,}
A is a direct bond, —O—, —S—, —NR⁹—, —CHR⁷— or —O—CHR⁷—,
with R⁹, when it is present, corresponding to an alkyl, an alkenyl or an alkynyl, it being possible for each of these groups to be substituted with an alkoxy, a haloalkoxy, an alkylthiol, a halogen or a phenyl unsubstituted or substituted with an alkyl, with a haloalkyl, with an alkoxyl, with a haloalkoxy, with an alkylthiol or with a halogen, or corresponds to hydrogen;
and R⁷ has the same definition as that given above for R⁹ or represents a hydroxyl; a halogen; a cyano; an acyl; alkoxy; a haloalkoxy or an alkylthiol;
A is linked to the 4-position of the benzene ring M; and
R⁶ is a phenyl or an aromatic heterocycle, unsubstituted or substituted with one or more substituents, which may be identical or different, and which may be selected from the following list: hydroxyl; halogen; cyano; acyl (preferably C(═O)R^{c}, —C(═S)R^{c} or —S(O)ₚR^{c}, with R^{c}=alkyl, haloalkyl, alkoxy, haloalkoxy, alkylthiol or phenyl unsubstituted or substituted with an alkyl, haloalkyl, alkoxy, haloalkoxy or alkylthiol); amine; alkylamine; dialkylamine; alkyl, haloalkyl, R^{a}O-alkyl, acyloxyalkyl, cyanooxyalkyl, alkoxy; haloalkoxy; alkylthiol; cycloalkyl (preferably cyclohexyl or cyclopentyl) unsubstituted or substituted with an alkyl, a haloalkyl, an alkoxy, a haloalkoxy or with an alkylthiol; and benzyl unsubstituted or substituted with an alkyl, a haloalkyl, an alkoxy, a haloalkoxy or with an alkylthiol.

The compounds of formula (I) which are still more especially preferred are those possessing the following characteristics, taken in isolation or combination:
R¹=H
R²=C₁-C₆ alkyl, preferably ethyl;
R³=C₁-C₆ alkyl, preferably methyl;
R⁴=C₁-C₆ alkyl, preferably methyl;
R⁵=C₁-C₆ alkyl, preferably methyl and R⁵ is linked to the carbon at C₅ of the benzyl ring M, with m=1;
A is linked to the carbon at C₄ of the benzyl ring M and represents —O—;
R⁶=aryl, preferably benzyl, advantageously substituted with at least one alkyl and/or with at least one halogen or at least one cyano group.

By way of example, the compounds (I) used are, inter alia:
N-ethyl-N-methyl-N′-[4-(4-chloro-3-trifluoromethylphenoxy)-2,5-dimethyl phenyl]imidoformamide,
and/or N-ethyl-N-methyl-N′-[4-(4-fluoro-3-trifluoromethylphenoxy)-2,5-dimethylphenyl]imidoformamide,
and/or N-ethyl-N-methyl-N′-[4-(4-cyano-3-trifluoromethylphenoxy)-2,5-dimethylphenyl]imidoformamide,
and the possible tautomers and salts, in particular addition salts with an acid or a base, which are pharmaceutically acceptable, of these compounds (I).

According to an advantageous embodiment of the invention, the antifungal medicament comprises at least one other antifungal compound (II).

Such an antifungal compound forms part of the compounds known to persons skilled in the art and is advantageously chosen from the following families of compounds:
azoles, such as bifonazole, butoconazole, clotrimazole, eberconazole, econazole, fenticonazole, fluconazole, itraconazole, ketoconazole, miconazole, oxiconazole, posaconazole, sulconazole, terconazole, tioconazole, voriconazole, zinoconazole;
polyenes, such as amphotericin B, nystatin;
allylamines and benzylamines, such as butenafine, naftifine, terbinafine;
thiocarbamates, such as tolnaftate;
candins, such as caspofungin, cilofungin;
nucleoside analogues, such as flucytosine;
sordarins;
polyoxines and nikkomycinrs, such as nikkomycins Z, J. pseudo J, PX, RZ, pseudo Z;
pradimicins, such as pradimicin A;
benanomycins;
aureobasidins;
UK-2A or UK-3A;
cationic peptides;
taken alone or as a mixture, and their possible tautomers and salts, in particular addition salts with an acid or a base, their lipid or liposomal formulations, which are pharmaceutically acceptable.

From the point of view of weight, it should be specified that in accordance with the invention, the mass ratio (I/II) is defined as follows:

| | | |
|---|---|---|
| | 0.02 | ≦I/II ≦ 50 |
| preferably | 0.1 | ≦I/II ≦ 20 |
| and still more preferably | 0.5 | ≦I/II ≦ 10. |

In the case where compound (II) is fluconazole or itraconazole (or one of their equivalents), it has been found that the mass ratio (I/II) is advantageously between 0.5 and 10.

The compound (I)/compound (II) ratio is defined as being the ratio by weight of these 2 compounds. The same applies to any ratio of 2 chemical compounds, which is subsequently measured in the present text, since a definition different from this ratio is not expressly given.

In the compositions according to the invention, the compound (I)/compound (II) ratio is advantageously chosen so as to produce a synergistic effect. The term synergistic effect, as understood in the present text, is defined in the examples at point 2.4.

As is evident from the preceding text, the preferred examples of synergistic combinations according to the invention will comprise compound (I), fluconazole and/or itraconazole, and their possible tautomers and addition salts with an acid or a base, as long as these equivalents are acceptable in the human or veterinary pharmaceutical field.

The compound I/compound (II) ratio ranges indicated above do not in any way limit the scope of the invention, but are rather mentioned as a guide, persons skilled in the art being entirely capable of carrying out additional trials in order to find other values of the apportioning ratio of these two compounds, for which a synergistic effect is observed.

According to a preferred feature of the invention, the quantity of active agents (I/II) present in the fungicidal compositions according to the invention is between 0.5 and 99% by weight.

Naturally, the antifungal medicaments according to the invention based on at least one compound (I) and at least one compound (II) may also comprise one or more other active products.

In addition to these additional active agents, the antifungal medicaments according to the invention may also contain any other excipient and/or auxiliary agent useful in pharmaceutical formulations.

As regards the presentations of the medicaments according to the invention, it should be indicated that they are appropriate for all known and suitable galenic forms in antifungal treatment. Thus, these medicaments may be provided in the form of formulations for administration orally, topically, intravenously or intraperitoneally.

As regards the preparation of compounds (I), reference may be made to international patent application WO-00/46184.

In the case of the preparation of the known synergistic compounds (II), these are prepared according to the usual pharmacopoea rules.

According to another of its objects, the invention relates to a method for controlling curatively or preventively, human or animal pathogenic fungi, characterized in that it consists in using an antifungal medicament as defined above.

The antifungal medicaments according to the invention usually contain from 0.5 to 99% of the combination of compound (I) and compound (II).

The optimum dose quite obviously depends on the type of pathogenic fungus to be treated and the seriousness of the infection.

The pathogenic fungi which are the targets of the antifungal medicament are in particular those taken as a whole comprising:
the group Deuteromycetes, and in particular Candida albicans, Candida tropicalis, Sporothrix schenckii, Coccidioides immitis;
the group Ascomycetes, and in particular Alternaria spp, Aspergillus fumigatus, Aspergillus flavus, Aspergillus niger, Aspergillus terreus, Cladocephalosporium spp, Cladosporium spp, Epidermophyton floccosum, Exophiala dermatitis, Fonsecaea compacta, Fonsecaea pedroso, Fusarium spp, Histoplasma capsulatum capsulatum, Histoplasma capsulatum buboisi, Microsporum spp, Paecilomyces spp, Paracoccidioides brasiliensis, Scedosporium apiospermum, Scedosporium prolificans, Scopulariopsis spp. Trichophyton rubrum;
the group Basidiomycetes, and in particular Cryptococcus neoformans neoformans, Cryptococcus neoformans gati.

Yet another subject of the invention relates to the use of at least one compound of formula (I) as defined above, taken alone or in combination with another antifungal compound (II), for the manufacture of an antifungal medicament.

Advantageously, the antifungal compound (II) is chosen from the families of antifungal compounds defined above.

Yet another subject of the invention relates to the use of a medicament as defined above, for the treatment of infections of fungal origin and in particular those caused by Candida albicans or Aspergillus fumigatus.

The following examples are given purely by way of illustration of the invention and do not limit it in any way.

### EXAMPLES

In Vitro Measurements of the Antifungal Activity of Various Compounds Used Alone or in Combination Against Candida albicans and Aspergillus fumigatus

### 1—Objective of the Trials

The objective of the trials is to test the efficacy of a compound of the arylamidine type, and two antifungal compounds of the family of azoles, fluconazole and itraconazole, already commercially available. These trials are aimed, in the first instance, at comparing the antifungal activity of the arylamidine type compound, taken alone, with that of azoles. Their aim is also to demonstrate the synergistic properties of the combinations of such compounds.

### 2—Materials and Methods

2.1—Strains and Media:

The following fungi were used for this study: Candida albicans strains IP 48.72 (ATCC 10231) and Aspergillus fumigatus strain IP 864.64 obtained from the Collection Nationale de Cultures de Microorganismes (CNCM) of the Institut Pasteur. The strains are cultured on Yeast Extract-Peptone-Dextrose (YEPD) agar medium comprising 0.5% yeast extract, 0.5% bactopeptone, 2% glucose and 2% agar at 30° C. and in the dark.

2.2—The Products Tested are:

COMPOUND (I.1): N-ethyl-N-methyl-N′-[4-(4-chloro-3-trifluoromethylphenoxy)-2,5-dimethylphenyl]imidoformamide.
COMPOUND (I.2): N-ethyl-N-methyl-N′-[4-(4-cyano-3-trifluoromethylphenoxy)-2,5-dimethylphenyl]imidoformamide.
COMPOUND (II.1): fluconazole.
COMPOUND (II.2): itraconazole.

All these compounds were prepared in a DMSO solution at a final concentration of 100 mg/ml. The stock solutions are stored at −20° C. up to the time of use.

2.3—Trial Medium:

The trials are carried out in RPMI 1640 medium with no sodium bicarbonate, but with L-glutamine buffered with 0.165 mol per litre of 3-[N-morpholino]propanesulphonic acid (MOPS), enriched (<<rich>>) or otherwise (<<minimal>>) with 2% glucose. The pH of this medium is adjusted to 7.0. The medium is sterilized by filtration (0.22 μm) and stored at 4° C. up to the time of use.

2.4—Measurement on Microtitre Plates:

All the antifungal tests are carried out on microtitre plates. The initial suspensions of spores are prepared in a sterile solution containing 0.85% NaCl, supplemented with Tween 80 at 0.01%. These initial suspensions are then diluted in the culture medium (RPMI 1640 enriched or otherwise with 2% glucose) to a final concentration of 10⁴ spores per ml. The measurements of viability of each inoculum are verified by subcultures of a volume of 300 μl on YEPD agar medium.

The antifungal compounds are tested in a range of concentrations ranging from 0.026 to 100 μg of active ingredient/ml. These antifungal compounds are then diluted in RPMI 1640 medium enriched or otherwise with 2% glucose. A final DMSO concentration of 0.2% is used throughout the measurements. Each trial is carried out on a series of dilutions of antifungal compounds, in duplicate. The antifungal dilutions (0.1 ml) and the fungal inoculum (0.1 ml) are added to each of the wells of the microtitre plate. The plates are then read with a spectrophotometer (ELX 800UV Bio-Tek Instruments, Inc) at a wavelength of 590 nm. The plates are read immediately after (t=0) and after 48 hours' incubation at 30° C. and in the dark. The optical density values obtained are correlated with fungal growth between the times t=0 and t=48 hours. The optical density values are used to calculate the percentage inhibition of growth for each concentration of antifungals by comparison with the control. The values are then used to plot a dose-response curve and the EC₅₀ value is determined for each fungus and each compound with the aid of the Grafit 5.0® software (Erithacus software Ltd).

The method which was used to measure the type of interaction existing between the antifungal compounds in the form of mixtures is the Wadley method.

In the Wadley approach, the dose-response curve for each of the compounds A and B, and for the mixture AB, is constructed. The EC₅₀ is calculated for each compound taken individually and for the mixture. If a and b are the absolute quantities of the compounds A and B in the mixture (a=1, b=1, a+b=2 under our conditions), the expected effective concentration (EC₅₀ₑₓₚ) may be calculated in the following manner:
EC₅₀ₑₓₚ=(a+b)/[a/EC_{50A})+b/(EC_{50B})

The Wadley approach may be used to estimate the type of interaction existing between the fungal compounds, regardless of their concentration. Its reliability is not dependent on the percentage inhibition. The type of interaction between two compounds is given by the level of interaction (LI) which corresponds to the ratio between the expected effective concentration (EC₅₀ₑₓₚ) and that observed (EC_{50obs}) of the mixture. The nature of the interaction obtained by the Wadley formula is presented in Table 1 (see U. Gisi, Synergistic interaction in fungicide mixtures, 1996. Phytopathology 86, 1273-1279) below.

| Level of interaction | Mathematical definition | Biological definition |
|---|---|---|
| <1 | Antagonist | |
| 1 | Additive | |
| >1 | Synergistic | |
| <0.5 | | Antagonist |
| 0.5-1 | | Additive |
| >1.15 | | Synergistic |

### 3—Results

TABLE 1
Efficacy in vitro of compound I.1, of
compound II.1 and of compound II.2
used alone against Aspergillus fumigatus
cultured on minimal RPMI 1640 medium (MM).

| | Dose (μg/ml) | | | | | | |
|---|---|---|---|---|---|---|---|
| % inhibition^{α} | 0.026 | 0.098 | 0.39 | 1.562 | 6.25 | 25 | 100 |
| Compound I.1 | 1 | 57.9 | 80.1 | 94.6 | 97.1 | 98.4 | 98.1 |
| Compound II.1 | 4.8 | 4.8 | 5.8 | 8.7 | 7.7 | 23.1 | 41.4 |
| Compound II.2 | 2.2 | 31.8 | 49.3 | 99.6 | 100 | 100 | 99 |

^{α}The percentage inhibition of growth is determined after 48 hours' incubation at 30° C. and in the dark.

TABLE 2
Efficacy in vitro of compound I.1, of
compound II.1 and of compound II.2
used alone against Aspergillus fumigatus
cultured on rich RPMI 1640 medium (RM).

| | Dose (μg/ml) | | | | | | |
|---|---|---|---|---|---|---|---|
| % inhibition^{α} | 0.026 | 0.098 | 0.39 | 1.562 | 6.25 | 25 | 100 |
| Compound I.1 | 2.24 | 23.1 | 51.1 | 97.4 | 98.1 | 98.1 | 97.4 |
| Compound II.1 | 11.2 | 20.7 | 25.9 | 27.6 | 28.4 | 48.3 | 46.6 |
| Compound II.2 | 18.6 | 47.5 | 88.6 | 99.2 | 100 | 100 | 100 |

^{α}The percentage inhibition of growth is determined after 48 hours' incubation at 30° C. and in the dark.

TABLE 3
Efficacy in vitro of compound I.1, of
compound II.1 and of compound II.2
used alone against Candida albicans
cultured on minimal RPMI 1640 medium (MM).

| | Dose (μg/ml) | | | | | | |
|---|---|---|---|---|---|---|---|
| % inhibition^{α} | 0.026 | 0.098 | 0.39 | 1.562 | 6.25 | 25 | 100 |
| Compound I.1 | 4.6 | 7 | 46.1 | 96.2 | 98.7 | 99.2 | 98.1 |
| Compound | 2.1 | 9.7 | 38.5 | 64.7 | 77.7 | 81.9 | 91.6 |
| II.1 | | | | | | | |
| Compound | 58.1 | 75.4 | 78.2 | 74.6 | 78.6 | 87.9 | 95.2 |
| II.2 | | | | | | | |

^{α}The percentage inhibition of growth is determined after 48 hours' incubation at 30° C. and in the dark.

TABLE 4
Efficacy in vitro of compound I.1, of
compound II.1 and of compound II.2
used alone against Candida albicans
cultured on rich RPMI 1640 medium (RM).

| | Dose (μg/ml) | | | | | | |
|---|---|---|---|---|---|---|---|
| % inhibition^{α} | 0.026 | 0.098 | 0.39 | 1.562 | 6.25 | 25 | 100 |
| Compound I.1 | 12.6 | 27.3 | 43.6 | 96.9 | 99.5 | 99.5 | 98.8 |
| Compound | 11.1 | 32.5 | 72.6 | 86.8 | 94 | 95.3 | 36.1 |
| II.1 | | | | | | | |
| Compound | 44.7 | 85.1 | 86.2 | 85.1 | 86.2 | 95.2 | 95.2 |
| II.2 | | | | | | | |

^{α}The percentage inhibition of growth is determined after 48 hours' incubation at 30° C. and in the dark.

TABLE 5
Efficacy^{α} in vitro of compound I.1,
of compound II.1 and of compound II.2
used alone against Aspergillus fumigatus and
Candida albicans cultured on minimal RPMI 1640
medium (MM) and on rich RPMI 1640 medium (RM).

| | EC₅₀ (μg/ml) | | | |
|---|---|---|---|---|
| | Aspergillus fumigatus | | Candida albicans | |
| Fungicide | MM | RM | MM | RM |
| Compound I.1 | 0.1 | 0.31 | 0.41 | 0.3 |
| Compound II.1 | 575^{β} | 125^{β} | 0.96 | 0.19 |
| Compound II.2 | 0.27 | 0.27 | 0.022 | 0.017 |

^{α}The percentage inhibition of growth is determined after 48 hours' incubation at 30° C. and in the dark.
^{β}These EC₅₀ values are extrapolated from the analysis of the dose-reponse curves obtained with the aid of the Grafit 5.0 ® software.

TABLE 6
Evaluation in vitro of the extent of
the interaction between compound I.1,
compound II.1 and compound II.2 using the Wadley
formula against Aspergillus fumigatus and
Candida albicans cultured on minimal RPMI
1640 medium (MM) and rich RPMI 1640 medium
(RM).

| | Aspergillus fumigatus | | Candida albicans | |
|---|---|---|---|---|
| Fungicide | MM | RM | MM | RM |
| Mixture I.1 + II.1 | | | | |
| EC_{50 obs} | 0.200 | 0.122 | 0.225 | 0.451 |
| EC_{50 exp} | 0.21 | 0.62 | 0.573 | 0.223 |
| L.I.^{α} | 1.06 | 5.09 | 2.54 | 0.494 |
| Mixture I.1 + II.2 | | | | |
| EC_{50obs} | 0.270 | 0.175 | 0.112 | 0.129 |
| EC₅₀ₑₓₚ | 0.15 | 0.292 | 0.042 | 0.032 |
| L.I. | 0.56 | 1.67 | 0.376 | 0.25 |

^{α}The level of interaction (L.I.) corresponds to the ratio of the expected effective concentration (EC₅₀ₑₓₚ) to the observed effective concentration (EC_{50obs}) of the mixture. The synergistic interaction is present when the level of interaction is greater than 1.5 (values in bold).

### 4—CONCLUSIONS

The various results obtained and presented above demonstrate the efficacy of compound I.1, whether on minimal RPMI 1640 medium (MM) or on rich medium (RM), against Aspergillus fumigatus and Candida albicans with EC₅₀ values between 0.1 and 0.5 μg/ml, and therefore having an activity equivalent to that of itraconazole (compound II.2) against Aspergillus fumigatus and an activity at least equivalent to that of fluconazole (compound II.1) against Candida albicans.

As regards the interactions between compounds, the results obtained by the Wadley method show that the combination of compound I.1 and fluconazole (compound II.1) exhibits surprising synergistic effects both on Aspergillus fumigatus and on Candida albicans. The antifungal medicament according to the invention therefore constitutes real progress in terms of improvement of the antifungal activity compared with the references on the market.

During a second study, compound I.2 according to the invention was tested. It is: N-ethyl-N-methyl-N′-[4-(4-cyano-3-trifluoromethylphenoxy)-2,5-dimethylphenyl]imidoformamide.

Compound (I.1) according to the invention and fluconazole (II.1) and itraconazole (II.2) were also tested in vitro on Candida albicans and Aspergillus fumigatus in enriched medium.

The experimental conditions are the same as previously described.

Results

TABLE 1
Efficacy^{α} in vitro of compounds I.1, I.2, II.1 and II.2,
used alone against Candida albicans
and Aspergillus fumigatus in RPMI
1640 medium enriched with 2% glucose.

| | EC₅₀^{χ} (μg/ml) | |
|---|---|---|
| | Candida albicans | Aspergillus fumigatus |
| Fungicide | IP 48.72^{β} | IP 864.64^{β} |
| Compound I.1 | 0.635 | 0.179 |
| Compound I.2 | 0.205 | 0.088 |
| Compound II.1 | 0.354 | 125 |
| Compound II.2 | 0.082 | 0.187 |

^{α}The percentage inhibition of growth is determined after 48 hours of incubation at 30° C. and in the dark.
^{β}Strains IP48.72 and IP864.64 served as references for these experiments.
^{χ}These EC₅₀ values are determined after analyses of the dose-reponse curves obtained by means of the Grafit 5.0 ® software.

On Candida albicans, the EC₅₀ of compound I.1 is 1.8 and 7.7 times higher than that of compounds II.1 and II.2, whereas compound I.2 shows better efficacy in vitro against Candida albicans than compounds I.1 and II.1.

On Aspergillus fumigatus (IP 864.64), the EC₅₀ of compound I.1 is about 700 times lower than that of compound II.1 and similar to that of compound II.2, whereas compound I.2 shows efficacy in vitro on Aspergillus fumigatus twice higher than that of compounds I.1 and II.2.

1. Antifungal medicament, characterized in that it comprises at least one compound of formula (I): 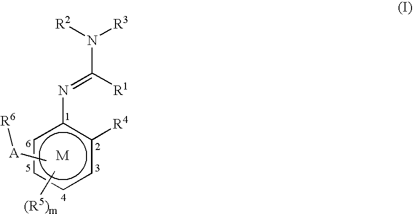
in which:
R¹ is an alkyl, an alkenyl, an alkynyl, a carbocyclic or heterocyclic monovalent group, it being possible for each of these groups to be substituted, or hydrogen;
R² and R³, which may be identical or different, are any one of the groups defined for R¹; a cyano; an acyl; —OR^{a} or —SR^{a}, with R^{a} corresponding to an alkyl, an alkenyl, an alkynyl, a carbocyclic or heterocyclic monovalent group, it being possible for each of these groups to be substituted, or R² and R³, or R² and R¹ may form together and with the atoms linking them, a ring which may be substituted;
R⁴ is an alkyl, an alkenyl, an alkynyl, a carbocyclic or heterocyclic monovalent group, it being possible for each of these groups to be substituted, a hydroxyl group; mercapto; azido; nitro; halo; cyano; unsubstituted or substituted acyl, amino; cyanato; thiocyanato; —SF₅; —OR^{a}; —SR^{a} or —Si(R^{a})₃;
m=0, 1, 2 or 3;
the optional R⁵ group or the optional R⁵ groups, which may be mutually identical or different, have the same definition as that given above for R⁴;
R⁶ is an unsubstituted or substituted carbocyclic or heterocyclic group; and
A is a direct bond, —O—, —S(O)ₙ—, —NR⁹—, —CR⁷═CR⁷—, —C≡C—, -A¹-, -A¹-A¹, —O-(A¹)ₖ—O—, —O—(A¹)ₖ—, -A³-, -A⁴-, -A¹O—, -A¹S(O)ₙ—, -A²-, OA²-, —NR⁹A²-, —OA²-A¹-, —OA²-C(R⁷)═C(R⁸)—, —S(O)ₙA¹-, -A¹-A⁴-, -A¹-A⁴-C(R⁸)═N—N═CR⁸—, -A¹-A⁴-C(R⁸)═N—X²—X³—, -A¹-A⁴-A³-, -A¹-A⁴-N(R⁹)—, -A¹-A⁴-X—CH₂—, -A¹-A⁴-A¹-, -A¹-A⁴-CH₂X—, -A¹-A⁴-C(R⁸)═N—X²—X³—X¹—, -A¹-X—C(R⁸)═N—, -A¹-X—C(R⁸)═N—N═CR⁸—, -A¹-X—C(R⁸)═N—N(R⁹)—, -A¹-X-A⁻-X¹—, -A¹-O-A³-, -A¹-O—C(R⁷)═C(R⁸)—, -A¹-O—N(R⁹)-A²-N(R⁹)—, -A¹-O—N(R⁹)-A²-, -A¹-N(R⁹)-A²-N(R⁹)—, -A¹-N(R⁹)-A²-, -A¹-N(R⁹)—N═C(R⁸)—, -A³-A¹-, -A⁴-A³-, -A²-NR⁹—, -A¹-A²-X¹—, -A¹-A¹-A²-X¹—, —O-A²-N(R⁹)-A²-, —CR⁷═CR⁷-A²-X¹—, —C≡C-A²-X¹—, —N═C(R⁸)-A²-X¹—, —C(R⁸)═N—N═C(R⁸)—, —C(R⁸)═N—N(R⁹)—, —(CH₂)₂—O—N═C(R⁸)— or —X-A²-N(R⁹)—
with
n=0, 1 or 2,
k=1 to 9,
A¹=—CHR⁷—,
A²=—C(═X)—,
A³=—C(R⁸)═N—O—,
A⁴=—O—N═C(R⁸)—,
X=O or S,
X¹=O, S, NR⁹ or a direct bond,
X²=O, NR⁹ or a direct bond,
X³=hydrogen, —C(═O)—, —SO₂— or a direct bond,
R⁷, which are mutually identical or different, each correspond to an unsubstituted or substituted alkyl, to a cycloalkyl or a phenyl, it being possible for each of these groups to be substituted, hydrogen, a halogen, a cyano, or an acyl;
R⁸, which are mutually identical or different, each correspond to an alkyl, an alkenyl, an alkynyl, an alkoxy, an alkylthio, it being possible for each of these groups to be substituted, a carbocyclic or heterocyclic monovalent group which may be unsubstituted or substituted, or hydrogen;
R⁹, which are mutually identical or different, each correspond to an unsubstituted or substituted alkyl, to a monovalent carbocyclic or heterocyclic group which may be unsubstituted or substituted, or to an acyl; or two R⁹ groups may form together, and with the atoms linking them, a 5-7-membered ring;
the group represented on the right side of the bond A is linked to R⁶;
or -A-R⁶ and R⁵ form together with the benzene ring M, a system of unsubstituted or substituted condensed rings;
and the possible optic and/or geometric isomers, tautomers and salts, in particular addition salts with an acid or a base, which are pharmaceutically acceptable, of the derivatives of formula (I)
and mixtures thereof.

2. Medicament according to claim 1, characterized in that:
R¹ is an alkyl, an alkenyl or an alkynyl, it being possible for each of these groups to be substituted with an alkoxy, a haloalkoxy, an alkylthiol, a halogen or a phenyl unsubstituted or substituted with an alkyl, with a haloalkyl, with an alkoxy, with a haloalkoxy, with an alkylthiol or with a halogen, or hydrogen;
R² and R³ which may be identical or different and which have the same definition as that given above for R¹ or which correspond to an alkoxy, an alkoxyalkyl, a benzyloxy, a cyano or an alkylcarbonyl;
R⁴ is an alkyl, an alkenyl or an alkynyl, it being possible for each of these groups to be substituted with an alkoxy, a haloalkoxy, an alkylthiol, a halogen or a phenyl unsubstituted or substituted with an alkyl, with a haloalkyl, with an alkoxy, with a haloalkoxy, with an alkylthiol or with a halogen; a hydroxyl; a halogen; a cyano; an acyl, an amine, a monoalkylamine, a dialkylamine or a phenyl unsubstituted or substituted with an alkyl, with a haloalkyl, with an alkoxy, with a haloalkoxy, or with an alkylthiol;
m=0 or 1;
when it is present, R⁵ is a group having the same definition as that given above for R⁴,
A is a direct bond, —O—, —S—, —NR⁹—, —CHR⁷— or —O—CHR⁷—,
with R⁹, when it is present, corresponding to an alkyl, an alkenyl or an alkynyl, it being possible for each of these groups to be substituted with an alkoxy, a haloalkoxy, an alkylthiol, a halogen or a phenyl unsubstituted or substituted with an alkyl, with a haloalkyl, with an alkoxy, with a haloalkoxy, with an alkylthiol or with a halogen, or corresponds to hydrogen;
and R⁷ has the same definition as that given above for R⁹ or represents a hydroxyl; a halogen; a cyano; an acyl; alkoxy; a haloalkoxy or an alkylthiol;
A is linked to the 4-position of the benzene ring M; and
R⁶ is a phenyl or an aromatic heterocycle, unsubstituted or substituted with one or more substituents, which may be identical or different, and which may be selected from the following list: hydroxyl; halogen; cyano; acyl; amine; alkylamine; dialkylamine; alkyl, haloalkyl, R^{a}O-alkyl, acyloxyalkyl, cyanooxyalkyl, alkoxy; haloalkoxy; alkylthiol; cycloalkyl unsubstituted or substituted with an alkyl, a haloalkyl, an alkoxy, a haloalkoxy or with an alkylthiol; and benzyl unsubstituted or substituted with an alkyl, a haloalkyl, an alkoxy, a haloalkoxy or with an alkylthiol.

3. Medicament according to claim 1, characterized in that:
R¹=H
R²=C₁-C₆ alkyl, preferably ethyl;
R³=C₁-C₆ alkyl, preferably methyl;
R⁴=C₁-C₆ alkyl, preferably methyl;
R⁵=C₁-C₆ alkyl, preferably methyl and R⁵ is linked to the carbon at C₅ of the benzyl ring M, with m=1;
A is linked to the carbon at C₄ of the benzyl ring M and represents —O—;
R⁶=aryl, preferably benzyl, advantageously substituted with at least one alkyl and/or with at least one halogen.

4. Medicament according to claim 3, characterized in that compound (I) is:
N-ethyl-N-methyl-N′-[4-(4-chloro-3-trifluoromethyl phenoxy)-2,5-dimethylphenyl]imidoformamide,
and/or N-ethyl-N-methyl-N′-[4-(4-fluoro-3-trifluoromethylphenoxy)-2,5-dimethylphenyl]imidoformamide,
and/or N-ethyl-N-methyl-N′-[4-(4-cyano-3-trifluoromethylphenoxy)-2,5-dimethylphenyl]imidoformamide,
and the possible tautomers and salts, in particular addition salts with an acid or a base, which are pharmaceutically acceptable, of these compounds (I).

5. Medicament according to claim 1, characterized in that it additionally comprises at least one other antifungal compound (II).

6. Medicament according to claim 5, characterized in that the antifungal compound (II) is chosen from the following antifungal families:
azoles, such as bifonazole, butoconazole, clotrimazole, eberconazole, econazole, fenticonazole, fluconazole, itraconazole, ketoconazole, miconazole, oxiconazole, posaconazole, sulconazole, terconazole, tioconazole, voriconazole, zinoconazole;
polyenes, such as amphotericin B, nystatin;
allylamines and benzylamines, such as butenafine, naftifine, terbinafine;
thiocarbamates, such as tolnaftate;
candins, such as caspofungin, cilofungin;
nucleoside analogues, such as flucytosine;
sordarins;
polyoxines and nikkomycins, such as nikkomycins Z, J, pseudo J, PX, RZ, pseudo Z;
pradimicins, such as pradimicin A;
benanomycins;
aureobasidins;
UK-2A or UK-3A;
cationic peptides;
taken alone or as a mixture, and their possible tautomers and salts, in particular addition salts with an acid or a base, their lipid or liposomal formulations, which are pharmaceutically acceptable.

7. Antifungal medicament according to claim 4, characterized in that the mass ratio (I/II) is defined as follows:

| | | |
|---|---|---|
| | 0.02 | ≦I/II ≦ 50 |
| preferably | 0.1 | ≦I/II ≦ 20 |
| and still more preferably | 0.5 | ≦I/II ≦ 10. |

8. Antifungal medicament according to claim 4, characterized in that the compound (I)/compound (II) ratio is chosen so as to produce a synergistic effect.

9. Antifungal medicament according to claim 8, characterized in that the compound (I)/compound (II) ratio is between 0.5 and 10.

10. Antifungal medicament according to claim 1, characterized in that it additionally comprises at least one pharmaceutically acceptable excipient.

11. Antifungal medicament according to claim 1, characterized in that it comprises from 0.5 to 99% of the combination of compound (I) and compound (II).

12. Use, for the manufacture of an antifungal medicament, of at least one compound of formula (I) 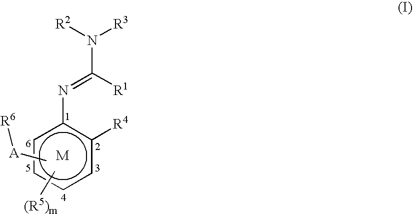
in which:
R¹ is an alkyl, an alkenyl, an alkynyl, a carbocyclic or heterocyclic monovalent group, it being possible for each of these groups to be substituted, or hydrogen;
R² and R³, which may be identical or different, are any one of the groups defined for R¹; a cyano; an acyl; —OR^{a} or —SR^{a}, with R^{a} corresponding to an alkyl, an alkenyl, an alkynyl, a carbocyclic or heterocyclic monovalent group, it being possible for each of these groups to be substituted, or R² and R³, or R² and R¹ may form together and with the atoms linking them, a ring which may be substituted;
R⁴ is an alkyl, an alkenyl, an alkynyl, a carbocyclic or heterocyclic monovalent group, it being possible for each of these groups to be substituted, a hydroxyl group; mercapto; azido; nitro; halo; cyano; unsubstituted or substituted acyl, amino; cyanato; thiocyanato; —SF₅; —OR^{a}; —SR^{a} or —Si(R^{a})₃;
m=0, 1, 2 or 3;
the optional R⁵ group or the optional R⁵ groups, which may be mutually identical or different, have the same definition as that given above for R⁴;
R⁶ is an unsubstituted or substituted carbocyclic or heterocyclic group; and
A is a direct bond, —O—, —S(O)ₙ—, —NR⁹—, —CR⁷═CR⁷—, —C≡C—, -A¹-, -A¹-A¹, —O-(A¹)ₖ—O—, —O-(A¹)ₖ—, -A³-, -A⁴-, -A¹O—, -A¹S(O)ₙ—, -A²-, OA²-, —NR⁹A²-, —OA²-A¹-, —OA²-C(R⁷)═C(R⁸)—, —S(O)ₙA¹-, -A¹-A⁴-, -A¹-A⁴-C(R⁸)═N—N═CR⁸—, -A¹-A⁴-C(R⁸)═N—X²-X³—, -A¹-A⁴-A³-, -A¹-A⁴-N(R⁹)—, -A¹-A⁴-X—CH₂—, -A¹-A⁴-A¹-, -A¹-A⁴-CH₂X—, -A¹-A⁴-C(R⁸)═N—X²-X³-X¹—, -A¹-X—C(R⁸)═N—, -A¹-X—C(R⁸)═N—N═CR⁸—, -A¹-X—C(R⁸)═N—N(R⁹)—, -A¹-X-A—X¹—, -A¹-O-A³-, -A¹-O—C(R⁷)═C(R⁸)—, -A¹-O—N(R⁹)-A²-N(R⁹)—, -A¹-O—N(R⁹)-A²-, -A¹-N(R⁹)-A²-N(R⁹)—, -A¹-N(R⁹)-A²-, -A¹-N(R⁹)—N═C(R⁸)—, -A³-A¹-, -A⁴-A³-, -A²-NR⁹—, -A¹-A²-X¹—, -A¹-A¹-A²-X¹—, —O-A²-N(R⁹)-A²-, —CR⁷═CR⁷-A²-X¹—C≡C-A²-X¹—, —N═C(R⁸)-A²-X¹—, —C(R⁸)═N—N═C(R⁸)—, —C(R⁸)═N—N(R⁹)—, —(CH₂)₂—O—N═C(R⁸)— or —X-A²-N(R⁹)—
with
n=0, 1 or 2,
k=1 to 9,
A¹=—CHR⁷—,
A²=—C(═X)—,
A³=—C(R⁸)═N—O—,
A⁴=—O—N═C(R⁸)—,
X=O or S,
X¹=O, S, NR⁹ or a direct bond,
X²=O, NR⁹ or a direct bond,
X³=hydrogen, —C(═O)—, —SO₂— or a direct bond,
R⁷, which are mutually identical or different, each correspond to an unsubstituted or substituted alkyl, to a cycloalkyl or a phenyl, it being possible for each of these groups to be substituted, hydrogen, a halogen, a cyano, or an acyl;
R⁸, which are mutually identical or different, each correspond to an alkyl, an alkenyl, an alkynyl, an alkoxy, an alkylthio, it being possible for each of these groups to be substituted, a carbocyclic or heterocyclic monovalent group which may be unsubstituted or substituted, or hydrogen;
R⁹, which are mutually identical or different, each correspond to an unsubstituted or substituted alkyl, to a carbocyclic or heterocyclic monovalent group which may be unsubstituted or substituted, or to an acyl; or two R⁹ groups may form together, and with the atoms linking them, a 5-7-membered ring;
the group represented on the right side of the bond A is linked to R⁶;
or -A-R⁶ and R⁵ form together with the benzene ring M, a system of unsubstituted or substituted condensed rings;
and the possible optic and/or geometric isomers, tautomers and salts, in particular addition salts with an acid or a base, which are pharmaceutically acceptable, of the derivatives of formula (I);
and mixtures thereof;
the said compound (I) being taken alone or in combination with another antifungal compound (II).

13. Use according to claim 12, characterized in that the antifungal compound (II) is chosen from the following antifungal families:
azoles, such as bifonazole, butoconazole, clotrimazole, eberconazole, econazole, fenticonazole, fluconazole, itraconazole, ketoconazole, miconazole, oxiconazole, posaconazole, sulconazole, terconazole, tioconazole, voriconazole, zinoconazole;
polyenes, such as amphotericin B, nystatin;
allylamines and benzylamines, such as butenafine, naftifine, terbinafine;
thiocarbamates, such as tolnaftate;
candins, such as caspofungin, cilofungin;
nucleoside analogues, such as flucytosine;
sordarins;
polyoxines and nikkomycins, such as nikkomycins Z, J, pseudo J, PX, RZ, pseudo Z;
pradimicins, such as pradimicin A;
benanomycins;
aureobasidins;
UK-2A or UK-3A;
cationic peptides;
taken alone or as a mixture, and their possible tautomers and salts, in particular addition salts with an acid or a base, their lipid or liposomal formulations, which are pharmaceutically acceptable.

14. Use of an antifungal medicament according to claim 1, for the treatment of Candida albicans infections.

15. Use of an antifungal medicament according to claim 1, for the treatment of Aspergillus fumigatus infections.
